(19) United States
(12) Patent Application Publication
(10) Pub. No.: US 2013/0013031 A1
(43) Pub. Date: Jan. 10, 2013

(54) CAPSULE PHOTOTHERAPY

(75) Inventors: Sharon Ben-Yehuda, Rechovot (IL); Ram Ben-Yehuda, Rechovot (IL)

(73) Assignee: PHOTOPILL MEDICAL LTD., Rechovot (IL)

(21) Appl. No.: 13/634,555

(22) PCT Filed: Mar. 17, 2011
(86) PCT No.: PCT/IL2011/000257

§ 371 Date: Sep. 13, 2012

Related U.S. Application Data
(60) Provisional application No. 61/314,762, filed on Mar. 17, 2010.

(51) Int. Cl. A61N 5/06

(52) U.S. Cl. 607/92

(57) ABSTRACT

The present invention provides a swallowable capsule suitable for providing phototherapy to a region of a patient's gastrointestinal (GI) tract, the capsule comprising one or more light sources emitting in the visible and/or NIR ranges and optical elements for shaping the light beam produced by said light sources, such that said light source(s) and said optical elements are capable of delivering an effective therapeutic dose to a target site within the GI tract. The present invention further provides a method for intraluminal phototherapy of the gastrointestinal tract using a swallowable capsule as described hereinabove.

### TECHNICAL FIELD

The present invention relates to an ingestible phototherapy device that may be used for treating diseases of the gastrointestinal tract, and in particular, for treating inflammatory bowel disease (IBD)

### BACKGROUND

Light therapy, conventionally referred to as “phototherapy”, comprises exposing living tissue to light to treat a disease of the organism or tissue. The exposure is typically provided in accordance with a particular protocol tailored to the disease that defines spectrum and intensity of light used to illuminate the tissue and total energy deposited in the tissue by the light. The light may be generated using any of various suitable light sources, such as lasers, light emitting diodes (LEDs) fluorescent lamps.

Phototherapy is generally applied to relatively easily accessible tissue regions, such as external regions of the skin and the mucosa lining the mouth or nose, and is used to treat acne, psoriases, eczema, vitiligo (in which damage to skin pigment cells results in white skin patches) and skin-based lymphoma, gingivitis, gum inflammations, oral ulcers, and allergic rhinitis.

Phototherapy for treatment of diseases of the gastrointestinal (GI) tract is generally not performed because of the relative difficulty in accessing GI tract tissue. International Patent Application Publication WO 2008/012701 is entitled “Capsule camera with variable illumination of the surrounding tissue”, and discloses an ingestible capsule that is primarily intended for in vivo imaging of the GI tract of mammalian subjects. While the publication also mentions the possible use of the disclosed capsule camera for the treatment of diseased regions of the GI tract, it provide only very sparse details of the way in which said camera may be used for this purpose or of the structural features which permit said use. An article entitled Autonomous Device for Photostimulation of the Gastrointestinal Tract Immunity by Sergey A. Naumov et. al. (Translated and prepared for Alpha Omega Laboratories, Inc. by ITI Holms, Moscow, Russia, Apr. 4, 2003) describes an ingestible capsule that is constructed from two metallic hemispheres which are separated by a connected with light-transmitting polymeric sleeve. While this publication does describe the use of an experimental capsule device for use in the stimulation of Immune System mediators, it does not describe a method for either treating lesions or for promoting tissue healing in. the GI tract. Furthermore, it does not disclose any means for focusing the light beam produced by the light source or for controlling the activation, de-activation or output of said light source.

In conclusion, the inventors are unaware of any prior art publications that disclose or teach devices that are suitable for performing routine capsule phototherapy of GI tract conditions in a safe and controllable manner. The present invention provides workable technical solutions that enable this deficiency to be overcome.

### SUMMARY OF THE INVENTION

The present invention is primarily concerned with an ingestible capsule device that is suitable for routine phototherapy of the GI tract mucosa, particularly in cases of IBD (such as ulcerative colitis and Crohn's disease). A key feature of the device of the present invention is the fact that all of the elements required for light irradiation of small and large bowel lesions, as well as means for controlling various key parameters associated with said irradiation are located “onboard”, that is, within the capsule device itself. As a result, the use of the capsule—and particularly the self-use by the patient herself/himself—is greatly facilitated by the fact that there is no requirement for the use of any external apparatus.

Thus, one aspect of the present invention relates to a phototherapy device for applying phototherapy to a patient's GI tract comprising a capsule, hereinafter also referred to as a “photopill”, which the patient swallows so that it passes through his or her GI tract, and in passing, illuminates defined annular regions of the GI tract with therapeutic light.

The present invention is thus primarily directed to a swallowable capsule device suitable for use in intraluminal phototherapy of the GI tract, wherein said capsule comprises one or more light sources, and optical elements for shaping the light beam produced by said light sources, such that said light source(s) and said optical elements are capable of delivering an effective therapeutic dose to a target site within the GI tract.

The aforementioned capsule generally has a shape similar to that of capsules produced for pharmaceutical use, with a smooth, rounded outline suitable for being easily swallowed by a human subject.

Preferably, the light beam produced by the light source(s) is shaped by the optical elements such that the emitted light is transmitted out of the capsule in a direction that is essentially perpendicular to the longitudinal axis of the capsule (i.e. approximately perpendicular to the direction of travel of the capsule). In this way, the emitted light will, in use, be directed perpendicular to the wall of the GI tract. Furthermore, the arrangement of the light sources and associated optical elements is such that the emitted light is transmitted outwards around the entire circumference of the capsule, thereby projecting an essentially circular narrow band of light that surrounds said capsule. This 360 degree illumination pattern may be created in a number ways including—but not limited to—the use of a plurality of light sources arranged around the circumference of the capsule, and the use of reflective and/or refractive optical elements in order to change the direction in which the emitted light beams travel.

Any suitable light sources may be used in order to work the present invention, including lasers and light emitting diodes (LEDs). Said light sources are selected such that they emit light/photon radiation centered at any desired wavelength within the visible or near infra-red (NIR) ranges. Typically, the wavelength used will be selected from one or more of the following ranges: 400-480 nm, 610-720 nm and 800-950 nm. Typical examples of emission wavelengths used include 440 nm (blue), 660 nm (red) and 850 nm (NIR). In some cases, it may be desirable to incorporate different sources emitting at different wavelengths in a single photopill device. As a result of the concentration of the light within a disc-like volume, as the photopill moves through the GI tract it provides concentrated therapeutic illumination to a relatively narrow annular band of the GI tract wall. Concentration of light from a photopill, in accordance with an embodiment of the invention, conserves optical energy provided by the photopill light source and improves efficiency with which the light is applied to walls of the GI tract. Other advantages of the present invention will become apparent as the description proceeds.

In another particularly preferred embodiment of the present invention, the ingestible capsule (photopill) further comprises means for determining its direction, speed of movement and location as it travels through the GI tract. In one embodiment, these means are provided by an accelerometer. In another embodiment, the means are provided in the form of an optical motion sensing system, generally comprising an illumination source and at least two photodetectors that are disposed within the capsule such that said photodetectors are capable of detecting light signals that were emitted by said illumination source and reflected back towards the capsule by an external structure, such as the intestinal wall.

In most embodiments of the present invention, the photopill capsule will further comprise control means such as one or more microprocessors (together with associated circuitry) for use in controlling all of the various activities performed by the capsule including (but not limited to) initial triggering of the power supply and timer clock, activation/deactivation of the therapeutic light source, calculation of capsular speed, direction and position from inputs provided by the motion detection system(s) and onboard timers, calculation of therapeutic light intensity and so on. A key advantage of the preferred embodiments of the capsule device of the present invention is that all of the elements required for therapeutic light radiation and for the control and regulation of all of the various parameters related to said radiation may be contained within a single small capsule, thereby obviating the need for ancillary control devices.

In an embodiment of the invention, the photopill comprises a light source and a controller which turns on the light source responsive to time measured by a timer to deliver therapeutic light following a predetermined delay time. The predetermined delay time is determined responsive to a rate at which the photopill travels through a patient's GI tract and location in the GI tract of a diseased region to be treated with phototherapy so that the light source turns on to deliver phototherapy substantially only when it approaches and is near to a length of the GI tract in which the diseased region is located. Controlling the photopill to begin illumination only when it approaches and is near to a length of the GI tract that includes the diseased region improves energy efficiency of the photopill and reduces an amount of energy that must be supplied to the photopill to deliver a desired dose of therapeutic light to the diseased region.

In some embodiments of the invention, the controller turns off the light source when the photopill leaves the length of GI tract including the diseased region and is not in a position to illuminate the diseased region. In some embodiments of the invention, the photopill is used to treat a plurality of different diseased regions of the GI tract that are located in different spatially separated lengths of the GI tract. For each diseased region the controller turns on the light source when it approaches the diseased region and is in a position to illuminate it with therapeutic light and subsequently, except for optionally a last diseased region, turns off the light after it leaves the diseased region.

It is noted that the present invention is, of course, not limited to delivering phototherapeutic light to only limited lengths of the GI tract and if desired, a photopill in accordance with an embodiment of the invention can be configured to deliver therapeutic light to substantially all of a patient's GI tract.

In some embodiments of the invention, the photopill is contained in a package and comprises a switch that is used to turn on various elements within the capsule, including (but not limited to) the timer (in order to initiate measuring time for determining the delay time and exposure period when the photopill is removed from the package in order to be swallowed by the patient), and/or means for determining the direction, speed of movement and location of the capsule (such as an accelerometer or optical motion-sensing system). Optionally, the switch comprises a magnetic “proximity” switch, which operates to turn on the timer when the photopill is distanced from a magnetic field generated by the package. Optionally, the switch comprises a mechanical switch which is triggered by removal of the photopill from the package. In a further embodiment, the initial triggering of the timer is effected operator-initiated squeezing or pressing of a mechanical switch element within the capsule.

In an embodiment of the invention, the phototherapy system comprises a set of photopills, each programmed with a different delay time and optionally a different exposure time. The set of photopills are used to provide phototherapy to different regions of a patient's GI tract while maintaining relatively low power consumption for each photopill.

In another aspect, the present invention provides a phototherapy system comprising an external beacon that transmits beacon signals such as radio or ultra sound beacon signals, and a photopill having a receiver for receiving the beacon signals. The beacon is located at a known location on a patient's body and locations of diseased regions of the patient's GI tract are correlated with characteristic features, such as for example frequency, polarization, and signal strength, of beacon signals transmitted by the beacon from the known location. After being swallowed by the patient, the photopill receives beacon signals and processes the signals to determine if the photopill is located in a region of the GI tract that is intended for phototherapy. If it determines it is located in such a region of the GI tract, the photopill turns on to illuminate the region with phototherapeutic light.

As mentioned hereinabove, in some embodiments of the invention, the photopill comprises an accelerometer for monitoring changes in speed with which the photopill travels through the GI tract. Optionally, changes in speed are used to control therapeutic light provided by the photopill. For example, assume it is advantageous to provide a given quantity of therapeutic light to a particular region of the GI tract per unit area of the region. In addition, it is also possible to program the photopill to provide different amounts of therapeutic light to different areas within the GI tract. Then intensity of therapeutic light provided by the photopill when traveling through the particular region is optionally controlled to be substantially proportional to the given quantity of therapeutic light to be delivered per unit area of the region times a speed determined from signals generated by the accelerometer i.e.—for faster travel speeds with smaller tissue exposure time, higher energy is required to maintain a constant desired dosage of therapeutic light. As disclosed hereinabove, other motion-detecting means (such as optical means) may be used in place of, or in addition to, an accelerometer.

In some embodiments of the invention, changes in speed are used to determine where the photopill is located in the GI tract. Optionally, location is determined by double integrating acceleration determined responsive to measurements by the accelerometer over time to determine distance traveled through the GI tract.

In some embodiments of the invention, a sudden change in speed indicated by acceleration measurements provided by the accelerometer is used to determine location. For example, material propagating through the GI tract moves more slowly in the Cecum than in the small intestine. As a result, a sudden decrease in speed of travel of the photopill indicated by accelerometer signals indicating sudden deceleration can be used to determine when the photopill reaches the Cecum.

There is therefore provided in accordance with an embodiment of the invention, a swallowable capsule for providing phototherapy to a region of a patient's gastrointestinal (GI) tract, the capsule comprising: at least one light source controllable to generate light for phototherapy; and a controller that turns on the at least one light source at a phototherapy start time to illuminate a portion of the GI tract that includes at least a portion of the region. Optionally, when the controller turns on the at least one light source at the phototherapy start time the capsule is located near to a position in the GI tract at which light from the light source can illuminate the region. Additionally or alternatively the near position is optionally within 10 cm of the region. Optionally, the near position is within 5 cm of the region. Optionally, the near position is within 2 cm of the region. In some embodiments of the invention, the controller comprises a timer.

Optionally, the phototherapy start time is a time determined relative to a clock-on time at which clock-on time the timer begins measuring time to determine the phototherapy start time. Optionally, the capsule is contained in a package and the clock-on time is a time set by removing the capsule from the package. The swallowable capsule optionally comprises a switch that is operated by removal of the capsule from the package to set the clock-on time. Optionally, the switch is magnetically operated. Optionally, the package comprises a magnet that generates a magnetic field and the switch is operated to set the clock-on time responsive to changes in the magnetic field at the capsule caused by removal of the capsule from the package.

In some embodiments of the invention, the switch comprises a push-button that is operated to set the clock-on time. Optionally, the push-button is mechanically operated to operate the switch. Optionally, the package comprises a protuberance and the push-button is depressed by the protuberance when the capsule is in the package and is released to operate the switch and set the clock-on time when the capsule is removed from the package.

In some embodiments of the invention, the controller operates the switch to set the clock-on time responsive to a change in a feature of the ambient environment of the capsule. Optionally, the feature is temperature. Additionally or alternatively the feature optionally comprises light. In some embodiments of the invention, the feature comprises pH.

In some embodiments of the invention, the phototherapy start time is a time delayed by a predetermined delay time from the clock-on time. Optionally, the predetermined delay time is determined responsive to location of the region and a speed with which the capsule travels through the GI tract to the region.

In some embodiments of the invention, the controller is configured to receive a beacon signal and determines the phototherapy start time responsive to a beacon signal generated by a beacon. Optionally, the beacon signal comprises a magnetic field. Optionally, the controller determines the phototherapy start time responsive to strength of the magnetic field.

In some embodiments of the invention, the beacon signal comprises an ultrasound signal.

In some embodiments of the invention, the beacon signal comprises an RF signal. Additionally or alternatively, the beacon signal is optionally characterized by frequency that is a function of direction relative to a location of the beacon. Optionally, the controlled determines the phototherapy start time responsive to the frequency.

In some embodiments of the invention, the controller determines the phototherapy start time responsive to intensity of the beacon signal.

In some embodiments of the invention, the controller turns off the at least one light source at a phototherapy stop time subsequent to the phototherapeutic start time following a predetermined exposure period during which it illuminates the GI tract with phototherapeutic light.

In some embodiments of the invention, the controller determines a plurality of phototherapy start times.

In some embodiments of the invention, a swallowable capsule comprises an accelerometer that generates acceleration signals responsive to acceleration of the capsule. Optionally, the controller receives the acceleration signals generated by the accelerometer. Optionally, the controller adjusts intensity of therapeutic light provided by the at least one light source responsive to the acceleration signals. Additionally or alternatively, the controller optionally determines a distance traveled by the capsule in a patient's GI tract responsive to the acceleration signals. Optionally, the controller turns on the at least one light source responsive to the determined distance.

There is further provided in accordance with an embodiment of the invention, a swallowable capsule for providing phototherapy to a region of the gastrointestinal (GI) tract of a patient, the capsule comprising: at least one light source controllable to generate light for phototherapy; and a light director that receives light from the at least one light source and concentrates the light within an essentially circular band shaped volume.

In another aspect the present invention is also directed to a method for intraluminal phototherapy of the gastrointestinal tract in a patient in need of such treatment comprising the steps of:
a) providing a swallowable capsule as disclosed hereinabove and described in more detail hereinbelow; and
b) oral administration of the capsule to said patient.

The term “intraluminal phototherapy” as used herein refers to the use of light-irradiation treatment in order to treat lesions and/or promote tissue healing from within the lumen of the GI tract. The methods provided in the present invention are thus applicable to the treatment and healing of conditions of all tissues accessible from the GI lumen including the intestinal mucosa and sub-mucosal tissues.

Preferably, the patient self-administers the capsule device.

In one preferred embodiment of this aspect of the invention, the method is used to treat IBD. The presently-disclosed method may be used to treat all types of IBD, including Crohn's disease, ulcerative colitis and indeterminate colitis.

In another preferred embodiment, the method of the invention is used to promote, encourage and accelerate healing of the intestinal mucosa and underlying tissues.

In one preferred embodiment of the invention, the method is used to treat lesions and/or promote healing of tissues that are present in the small intestine.

### BRIEF DESCRIPTION OF FIGURES

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 schematically shows a photopill in accordance with an embodiment of the invention.

FIG. 2 schematically shows programming the photopill shown in FIG. 1, in accordance with an embodiment of the invention.

FIGS. 3A and 3B schematically show photopills similar to the photopill shown in FIG. 1 being used to treat diseased regions of a patients GI tract, in accordance with an embodiment of the invention.

FIG. 4 schematically shows the photopills shown in FIGS. 3A and 3B contained in a package that turns on their internal electronic circuitry, which initiates clocks to time delay and exposure times when they are removed from the package, in accordance with an embodiment of the invention.

FIG. 5 schematically shows another photopill whose timer is turned on to time delay and exposure times when it is removed from a package in which it is contained, in accordance with an embodiment of the invention.

FIG. 6 schematically shows a photopill fitted with a controller and an antenna suitable for receiving a beacon signal.

FIG. 7 schematically shows the photopill shown in FIG. 6 being used to provide phototherapy to a diseased region of a patient's GI tract in accordance with an embodiment of the invention.

FIG. 8 schematically shows the photopill providing phototherapy to a diseased region responsive to directional beacon signals in accordance with an embodiment of the invention.

FIG. 9 schematically illustrates the very wide angle illumination pattern produced by a typical silicon phototherapy light source.

FIG. 10 schematically illustrates the effect of placing re-shaping optics in front of a typical silicon phototherapy light source.

FIG. 11 depicts a capsule device of the present invention fitted with a ring-shaped lens element in front of silicon light sources elements.

FIG. 12 illustrates another embodiment of the present invention, in which a small number of light sources are located on the end face of the capsule.

FIG. 13 shows the internal arrangement of the light source and associated optical elements in the embodiment depicted in FIG. 12.

FIG. 14 depicts an embodiment of the present invention in which initial triggering of the capsule is achieved by squeezing a metal ring.

FIG. 15 illustrates the general features of an optical movement sensing system that is used in certain embodiments of the present invention.

FIG. 16 provides further details of the optical movement sensing system depicted in FIG. 15.

FIG. 17 depicts a typical reflected light signal as detected by the photodetectors in the optical movement sensing system used in some preferred embodiments of the present invention.

### DETAILED DESCRIPTION

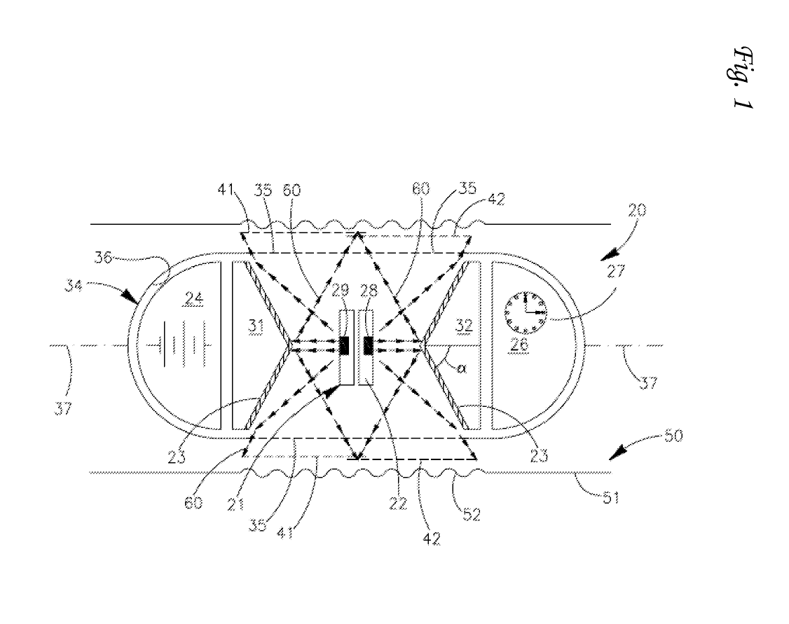

FIG. 1A schematically shows a cross section view of a photopill 20 providing phototherapy to a diseased portion of wall 51 of GI tract 50 of a patient, in accordance with an embodiment of the invention. The diseased portion of GI wall 51 is indicated by a wavy portion 52 of the wall.

Photopill 20 comprises, optionally two, light sources 21 and 22 and light directors 31 and 32 housed inside a capsule housing 34 having an external wall 36 and an axis 37, in accordance with an embodiment of the invention. Capsule housing 34 is characterized by dimensions suitable for swallowing and passage through the GI tract. By way of example, photopill 20 is optionally about 20 mm long and has a circular cross-section diameter of about 10 mm. Light sources 21 and 22, and light directors 31 and 32, are optionally located in a central portion of capsule housing 34 that is surrounded by a region, hereinafter referred to as “window”, represented by a dashed portion 35 of wall 36 that is transparent to light provided by light sources 21 and 22. Light directors 31 and 32 receive light, represented by arrows 60, from light sources 21 and 22 respectively and direct the received light to pass through window 35 of wall 36 to illuminate annular regions, indicated by dashed lines 41 and 42 respectively, of GI tract 50. Annular regions 41 and 42 define disc shaped volumes in which light from light sources 21 and 22 respectively are concentrated. The photopill comprises a power supply 24 and controller 26 optionally located at opposite ends of capsule housing 34. In an embodiment of the invention, controller 26 comprises a timer 27 and controls light sources 21 and 22, as described below, responsive to clock signals provided by the timer.

Light sources 21 and 22 may comprise any light source, such as a laser or LED, suitable for providing light in accordance with a desired phototherapy protocol for treating a diseased region of GI tract 50. By way of example, in photopill 20, light sources 21 and 22 are shown as LEDs having light emitting junctions 29 and 28 located on axis 37. In some embodiments of the invention light sources 21 and 22 provide light in substantially same wavelength bands. In other embodiments, light source 22 provides light in a wavelength band different from a wavelength band in which light source 21 provides light.

By way of example, assume that diseased portion 52 of GI tract 50 is afflicted with, and photopill 20 is configured to provide phototherapy for, inflammatory bowel disease (IBD). To treat IBD in diseased portion 52 in accordance with an embodiment of the invention, light source 21 optionally provides light in a wavelength band having width of about 30 nm centered at about 660 nm (nanometers) and light source 22 optionally provides light in a wavelength band of bandwidth about equal to 30 nm centered at about 850 nm. Advantageously, intensity of light emitted by each light source 21 and 22 is such that between about 0.1 to about 1 joules of phototherapeutic light at each wavelength band is deposited per cm² of GI tract tissue afflicted with IBD. It is to be recognized that light sources emitting at other wavelengths in the visible spectrum (e.g. 440 nm) and near infrared (NIR) spectrum (e.g. 850 nm), or any combination thereof, may also be used to work the present invention.

It has been found by the present inventors that higher light dosage levels do not necessarily result in more efficient phototherapy of GI tract lesions. On the contrary, in many cases it has been found that lower dosage levels yield better treatment results than higher light irradiation levels. However, it is also to be recognized that a minimum effective dosage level also exists.

Consequently, an effective intestinal phototherapy capsule should be able to deliver the correct amount of treatment energy, which is within the effective dosage zone for the device. A simple approach comprising swallowing a light source with a battery will not be effective and might even be harmful. Accurate light dose control is therefore of the utmost importance, in order to avoid the following undesirable scenarios:
a) Under dosage will have no effect.
b) Over dosage will have no effect and might cause local damage (heating the tissue).
c) The power source (e.g. miniature battery) may be exhausted before an effective dose has been provided to the tissue.
d) Coverage of the entire intestine wall's circumference is often required in order to obtain a satisfactory therapeutic approach.

It has been found that the light irradiation dose (commonly expressed in units of Joule per square centimeter) is affected by exposure time (seconds) and radiation power (Watts per square centimeter).

The main factors that influence dose delivery to the tissue include:
Changes in illumination pattern projected on the intestine wall, thereby causing changes in the actual delivered dose.
Changes in intestinal travel time, thereby causing changes in actual delivered dose due to change in exposure time.

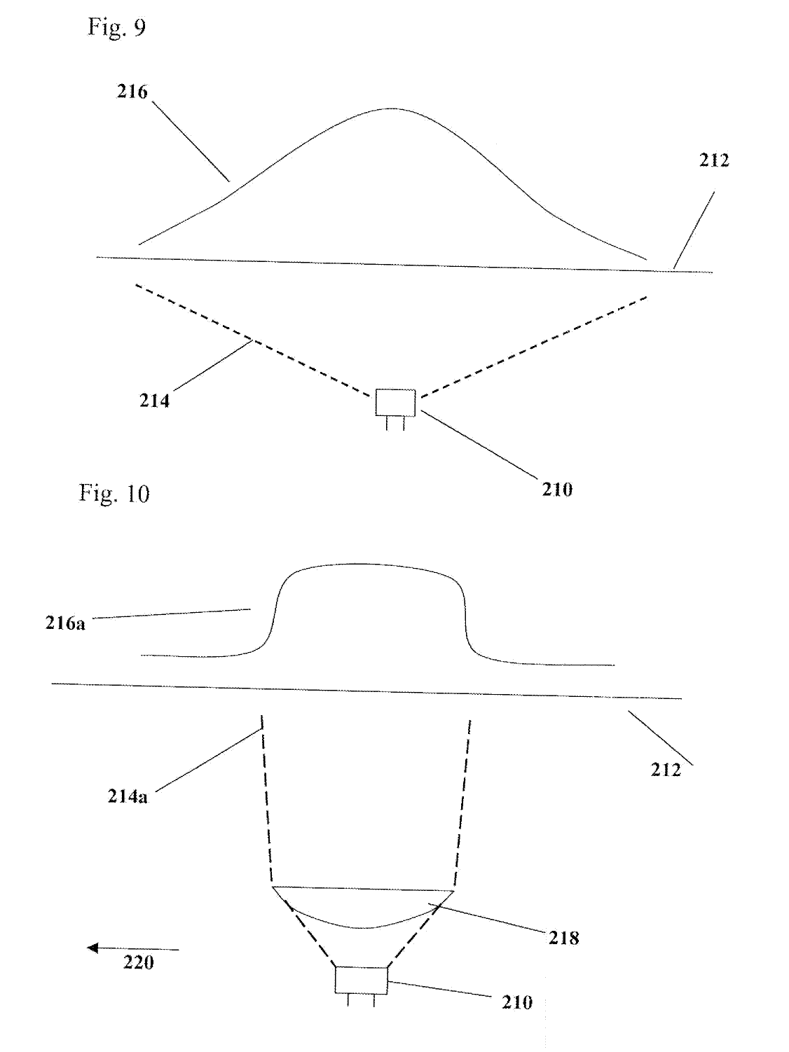

Due to the size of a swallowable capsule and the power it may require to support the phototherapy throughout the intestinal travel, a silicon based phototherapy source is a highly practical solution to the problem of meeting the aforementioned requirements. Such a silicon based source (for example HSDL-4400 manufactured by LIGHTON) has a very wide angle of illumination (typically 110-140 degrees) which forms an illumination pattern on the intestine wall which decays along the longitude axis (travel axis) of the capsule, as depicted in FIG. 9.

Thus, In FIG. 9, a silicon phototherapy source 210 is shown irradiating outwards towards the intestinal wall 212. Due to its wide angle of radiation 214 and due to the fact that radiation intensity is reducing in proportion to the square distance from the source, the radiation pattern absorbed by the intestinal wall 216 changes drastically. At some points in the pattern, very high energy levels can be found (adjacent to the light source) while at other points, very low, sub-minimal threshold energy levels are found.

For a silicon phototherapy based source to be effective, it should produce a known and measurable power level that can be compared against the required “dose response curve” to verify that it delivers the correct amount of therapeutic energy to the tissue.

One solution is to use an off-the-shelf silicon emitter with pre-manufacture optical lens incorporated into the component or alternatively, embedding the lens into the capsule's outer shell.

Although narrowing the beam angle prevents the delivery of a sub-threshold and ineffective treatment dose to the intestine wall it also creates another problem, namely that of delivering sufficient therapeutic power to the entire intestine circumference in the region of the GI tract that is being treated.

As mentioned hereinbefore, one of the requirements of a preferred embodiment of the phototherapy intestinal capsule of the present invention is to deliver light therapy to the entire circumference of the intestinal segment being treated (i.e. 360 degree irradiation around the capsule). It may thus be seen that simply narrowing down the angle of the silicon illuminator will, in many instances, be impractical since the increased number of light sources that will be required to be placed around the external circumference of the capsule will not physically fit within the confines of a capsule that is small enough to be swallowed.

In one preferred embodiment of the invention, a solution to this problem is provided by means of using beam-shaping optics which are designed to create a 360 degree irradiation coverage around the capsule's circumference while providing only a narrow irradiation pattern along the capsule's longitudinal axis (travel axis). Such a beam is formed with a specified width of its radiation pattern on the intestinal wall, where power density and therefore, dosage delivery, is more uniform and measurable.

In one preferred embodiment of this aspect of the invention, the silicon illuminators used (e.g. HIRTLB2-4G manufactured by Huey Jann Electronics Co.) have a wide radiation pattern, and are placed around the entire circumference of the capsule. A minimum of 3 such illuminators can be used to obtain 360 degrees coverage around the capsule's circumference. Re-shaping optics, shaped like a ring and placed over the light sources, concentrate the beam only along its longitudinal axis into a narrower beam.

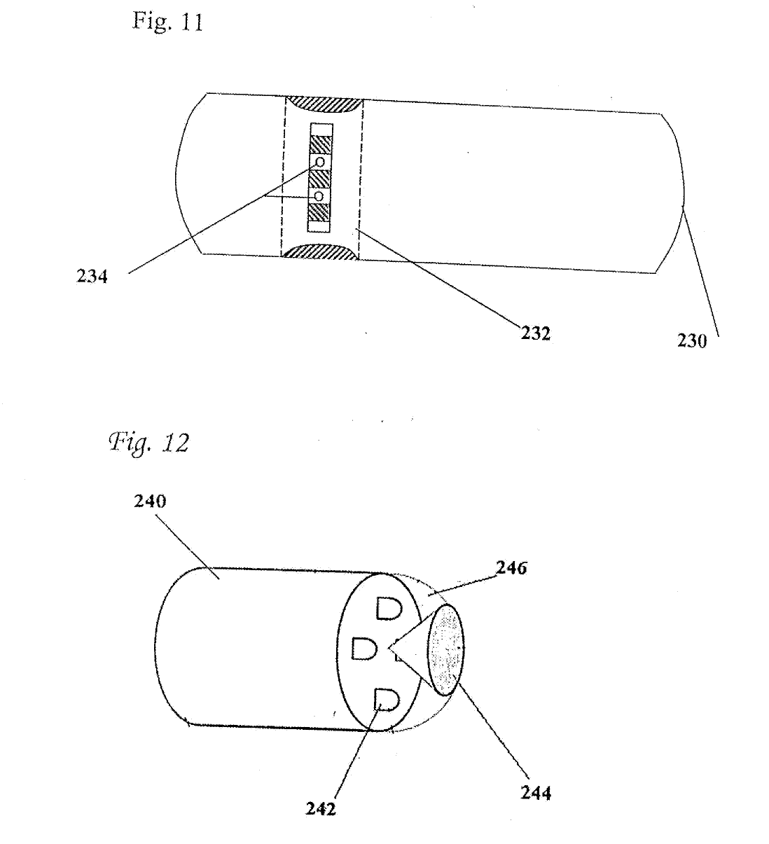

As seen in FIG. 10 the irradiation beam 214a created on the intestinal wall using this embodiment is formed in the shape of a ring having a unified energy density 216a. This irradiation pattern is achieved by means of a convex lens 218 placed between light source 210 and intestinal wall 212. The direction of travel of the photopill capsule is indicated by arrow 220. Further details of this embodiment are provided in FIG. 11, which shows a photopill capsule 230 fitted with a circumferentially arranged annular-shaped lens element 232 (“optical ring”) overlaying silicon illumination sources 234.

In an alternative preferred embodiment, instead of the illumination sources being arranged in a circumferential manner, they are disposed such that they point either in a forward or backward direction along the longitudinal axis of the device, as shown in FIG. 12. In this embodiment of the capsule device 240, fewer, but stronger, light sources 242 are needed in order to produce the required energy output. In order to achieve the desired illumination pattern on the intestinal wall, a funnel-shaped reflective optical element 244 is used to redirect the generated beam radially-outwards.through an optically-transparent dome 246.

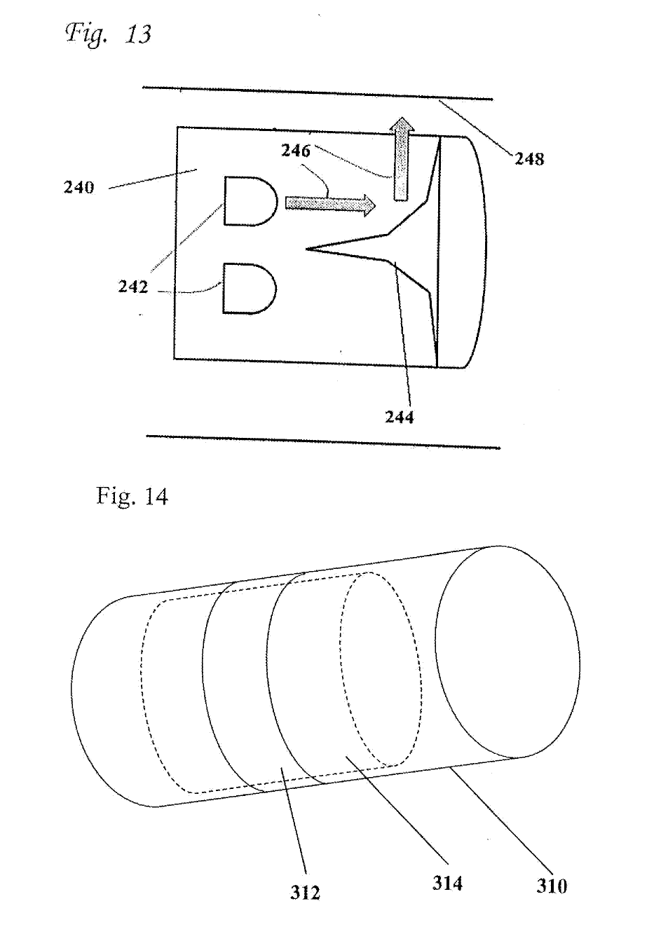

The internal arrangement of this embodiment is illustrated in more detail in FIG. 13, in which the arrows 246 indicate the change in direction of the light beam generated by light source 242, when said beam is incident on the reflective element 244. In this way, the light beam is projected radially outward onto the internal wall of the GI tract 248.

The embodiments that have been discussed thus far achieve delivery of the desired dosage levels to the intestinal tissue by means of controlling the illumination pattern produced by the light sources. However, as mentioned hereinabove, it also possible to obtain the desired dosage levels by a different approach, namely by controlling the capsule's intestinal travel time, thereby causing changes in actual delivered dose due to change in exposure time.

While certain factors involved in exposure time (mainly biological elements determining intestinal motility) are unable to be controlled, it is possible to control other factors. Thus, in one preferred embodiment of the present invention, the photopill device incorporates mechanisms for controlling the intensity of the emitted light in response to changes in the velocity of said device as it travels through the GI tract. These mechanisms will be described in further detail hereinbelow.

Another embodiment of the optical elements that may be used to generate the desired irradiation pattern is presented in FIG. 1, which shows light directors 31 and 32, which may be constructed of any suitable reflective material. In this embodiment of the invention, each light director 31 and 32 comprises a conical reflector 23 having an axis of rotation coincident with axis 37 of photopill 20. Relative locations of annular illuminated regions 41 and 42, whether the regions overlap or don't overlap, and if they overlap, by how much they overlap, are a function of spatial configuration of LEDs 21 and associated reflectors 23, a cone angle α of conical reflectors 31 and 32, and a diameter of a portion of GI tract 50 illuminated. For some configurations and GI tract diameters, and as shown in FIG. 1, annular illuminated regions 41 and 42 overlap over a relatively small area.

It is noted that light directors are not limited to simple conical light reflectors having a single cone angle, for which all surface elements make a same “inclination” angle with respect to an axis of the cone that is equal to a compliment of the cone angle. A conical light reflector may comprise a surface having regions that make different angles with respect to the cone axis. By way of example, a cone reflector in accordance with an embodiment of the invention optionally comprises surface regions having an inclination angle that gradually increases from about 30° to about 60° with distance of the surface region from the cone axis. Light directors are of course also not limited to cone reflectors or reflectors, and may for example, comprise any of various types of reflecting element, lenses, diffraction gratings, and/or light pipes and direct light from a single or a plurality of light sources to illuminate an annular region of a GI tract.

In one preferred embodiment of the invention, controller 26 turns on and turns off light source 21 and/or 22 responsive to clock signals provided by timer 27 so that photopill 20 provides phototherapy only to a region or regions of a patient's GI tract, for example diseased region 52 of GI tract 50, for which phototherapy is intended. Optionally, photopill controller 26 is programmable with at least one phototherapy start time and at least one phototherapy stop time to control when the controller turns on and turns off light source 21 and/or 22. A phototherapy start time is a time at which the controller turns on light source 21 and/or 22 to initiate phototherapy of a region of the patient's GI tract with light from light source 21 and/or 22, and a phototherapy stop time is a time at which it turns off light source 21 and/or 22 to terminate phototherapy provided by light source 21 and/or 22.

Phototherapy start and stop times are measured relative to a “clock-on” time, at which timer 27 begins clocking time to determine phototherapy start and stop times. Clock-on time is a time associated with a time at which the patient swallows photopill 20 to undergo phototherapy provided by the photopill.

Optionally, clock-on time is a time substantially equal to a time at which the patient swallows photopill 20. In some embodiments of the invention, clock-on time is a time characterized by a predetermined time difference relative to a time at which a patient swallows the photopill. For example, clock-on time may be set after swallowing photopill 20 by a time it takes gastric acids to dissolve an insulator and close thereby a circuit that sets the clock-on time.

In some embodiments of the invention, clock-on time is determined by a change in an ambient environment of the photopill associated with using the photopill for phototherapy. The change causes setting of the clock-on time. For example, photopill 20 is optionally stored at a temperature less than body temperature. When swallowed, body heat raises the temperature of the photopill and the temperature change sets the clock-on time. In some embodiments, photopill 20 comprises a pH monitor, and clock-on time is set responsive to a change in pH, such as a possible change in pH detected when the photopill is swallowed by a patient and comes in contact with the patient's saliva. In some embodiments of the invention, the photopill comprises electrodes that are exposed to liquid or tissue in a patient's mouth or GI tract after the photopill is swallowed. Changes in direct current (DC) resistance or alternating current (AC) impedance between the electrodes are used to set clock-on times.

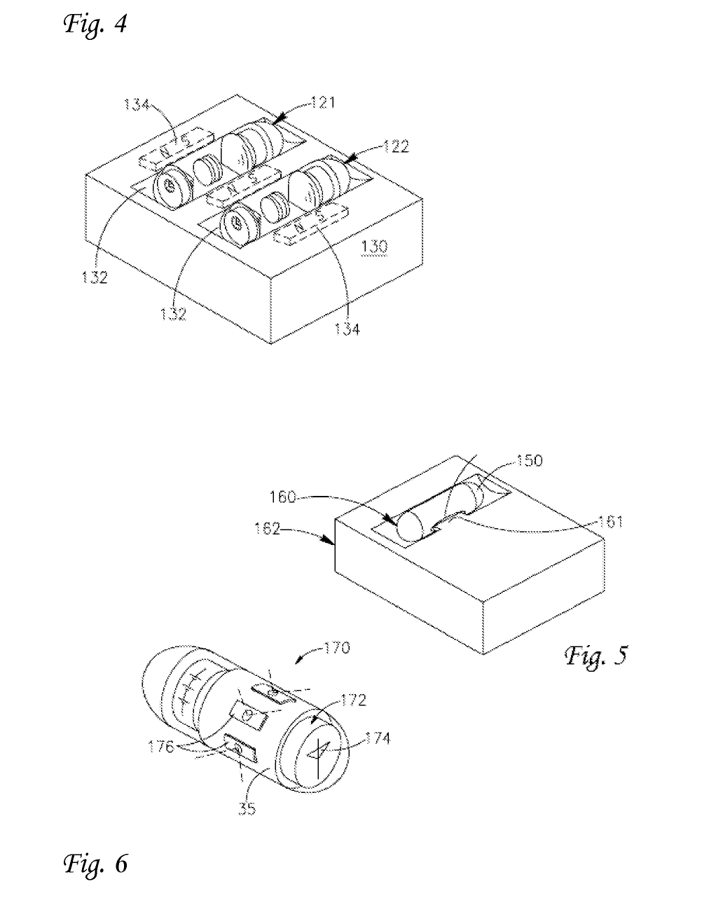

In some embodiments of the invention, clock-on time is a time at which the patient removes photopill 20 from a package in which it is contained. An act of removing the photopill from the package determines the clock-on time. Embodiments of the invention in which removal of photopill 20 from a package sets the clock-on time are discussed below with reference to FIG. 4 and FIG. 5. However, before beginning that discussion, a further embodiment of an activation mechanism will now be described with reference to FIG. 14, which illustrates a capsule device 310 fitted with an external metal ring 312. Immediately prior to use, the patient (or a medical attendant) squeezes said metal ring such that it is caused to make electrical contact with an annular metal plate 314 located interiorly to said ring. This circuit closure then connects the battery to the electronic circuitry within the capsule, thereby initiating its operation. In one preferred embodiment of this type, an indicator light contained within the capsule, or on its surface, is illuminated in order to indicate that the capsule's circuitry has been activated.

Returning now to our more general discussion of activation mechanisms (with reference to FIGS. 4 and 5), a phototherapy start time is determined responsive to location of a diseased region in the GI tract of a patient who is to undergo phototherapy so that at least one of light source 21 and light source 22 is turned on at a time following clock-on time that it takes photopill 20 to travel through the GI tract to the diseased region. A phototherapy stop time associated with the phototherapy start time is optionally determined by an extent of the diseased region and a time it takes photopill 20 to travel through the diseased region so that light sources 21 and 22 are turned off after the photopill leaves the diseased region and is no longer in a position to illuminate the diseased region.

Location of a diseased region and its extent may be determined using any of various medical imaging modalities, such as capsule endoscopy, magnetic resonance imaging (MRI), X-ray computerized tomography (CT) and ultrasound imaging, or by simple patient indication, “it hurts here!”, and/or professional palpation.

The travel time of photopill 20 may be estimated using data provided by various studies such “Compartmental Transit and Dispersion Model Analysis of Small Intestine Transit Flow in Humans”, by Lawrence X. Yu, John R. Crison and Gordon L. Amidon; International Journal of Pharmaceutics, Vol 40; 1999 and “Relationship of Gastric Emptying and Volume Changes After Solid Meal in Humans”; by Duane D. Burton, H. Jae Kim, Michael Camilleri, Debra A. Stephens, Brian P. Mullan, Michael K. O'Connor, and Nicholas J. Talley; Am J Physiol Gastrointest Liver Physiol 289, 2005. Optionally, travel times for a given patient are estimated from measurements of travel times of objects through the GI tract of the patient. For example, an acoustically reflective “calibration photopill” may be swallowed by the patient and progress of the calibration photopill through the patient's GI tract measured using ultrasound sensors.

As explained hereinabove, measurements of the movement of the photopill capsule may be used to calculate its average speed and location within the GI tract. In turn, these parameters may be used to control the start and stop times for the therapeutic illumination of the target tissue. In some particularly preferred embodiments of the present invention, both the determination of the photopill movements and the calculation of its speed and location from this determination, as well as control of the light source are performed by elements contained onboard, within the device itself, thereby obviating the need for additional externally-placed devices. In some embodiments of the invention, the device comprises an onboard accelerometer in order to measure the distance traveled along the GI tract. In these embodiments, phototherapy start and stop times are determined by onboard processing means (as will be described in more detail hereinbelow) using as their input the distance traveled by the photopill through the GI tract as determined from the accelerometer output and distance of the diseased region from a known location in the GI tract. Optionally “travel distance” is determined by double integrating acceleration measurements provided by the accelerometer. For example, if the diseased region is located between 1.5 and 1.6 meters from the mouth, a phototherapy start time is a time at which the double integrated acceleration is equal to about 1.5 meters. A subsequent phototherapy stop time is a time at which the double integrated acceleration is equal to about 1.6 meters.

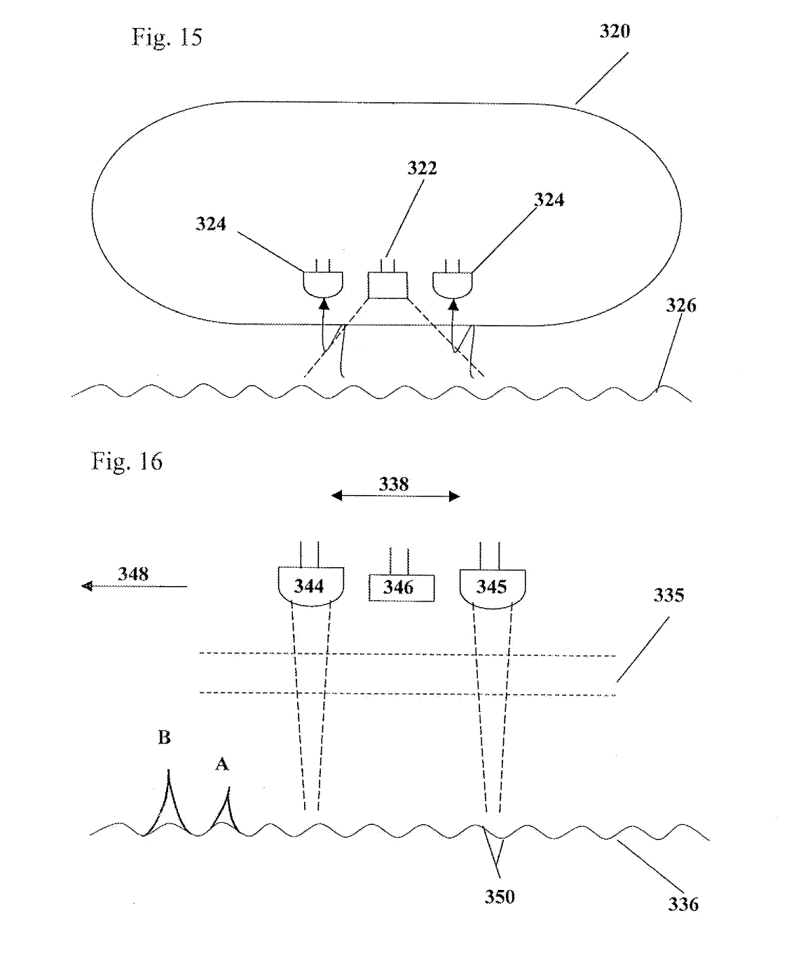

In other preferred embodiments, the onboard means for determining and measuring the movements of the photopill comprise optical motion sensing means (similar, in principle to an optical mouse of the type commonly used to position a cursor on a computer screen). In these embodiments, as depicted in FIG. 15, the capsule 320 includes an optical movement sensing element which comprises an illumination source (“illuminator”) 322, directed outwards through the side of the capsule by way of its transparent shell, and two or more optical photodetectors 324 also directed to a point outside the capsule which are responsive to optical signals reflected back from the intestinal walls 326.

The illuminator, such as the SMD LED by SunLED (model XZMDKT53W-6) is placed in a way which directs its light onto the small intestine wall to the same location that the photo detectors are focused on. A pair of photo detectors (such as the SMD HSDL-54xx series of PIN photodetectors by LiteON) are placed in a way that they are both focused to the same distance away from the capsule but each is placed in pre-defined distance (such as 5 mm) from the other, along the longitude axis of the capsule.

Referring now to FIG. 16, when the optical elements are positioned as described above, while the capsule is moving along the small intestine 336, the light emitted by the illuminator 346 (via transparent shell 335) and reflected back from the small intestine wall, is detected by the detectors 344 and 345. The focus area of detector 345 on the intestinal wall is indicated by numeral 350. Since the detectors are located in a pre-defined distance 338 from each other, a slightly different reflection will be obtained by the two detectors. As the capsule progresses (in the direction indicated by arrow 348), the detected signals will differ in phase to a degree which is dependent on the speed of capsule movement. FIG. 16 also shows the output of photodetectors 344 and 345 when the capsule is moving within the intestine. Reflections from the intestine wall (such as A and B) are detected by the photo-detectors according to the direction of movement. In the example demonstrated in this figure, the reflection A and then B will first be detected by the detector located closest to the leading edge of the capsule (i.e. detector 344).

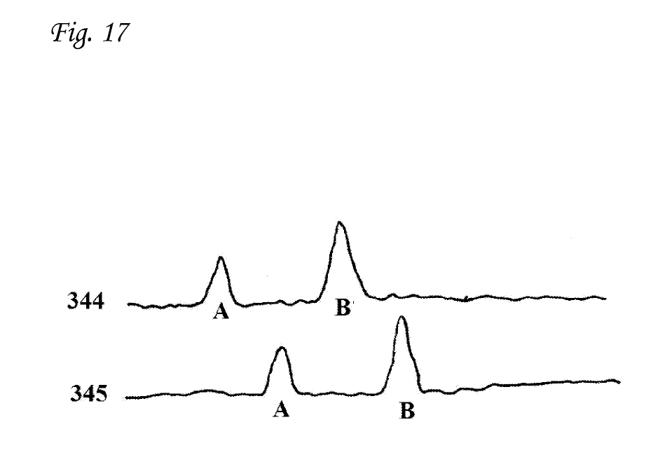

The output of the pair of detectors is also graphically illustrated in FIG. 17. As may be seen from this figure, the detection by the leading detector 344 occurs earlier than the detection by detector 345 that is located closer to the trailing edge of the device.

It may thus be appreciated that once the identity of the first detector that detects a reflected signal is known, the direction of movement can be obtained. Also, Since the detectors are placed in a pre-defined distance from each other, the speed of movement can be obtained by calculating the time elapsed between detection of a specific event by detector #1 and the detection of the same event by detector #2 and dividing the distance between the detectors by the detection difference time.

The values of movement direction and speed can be easily obtained by several methods, such as phase detector or by software techniques such as cross correlation between the two detectors outputs.

Following the determination of the speed and direction of movement of the device, its location within the intestine can be determined by integrating the speed over time.

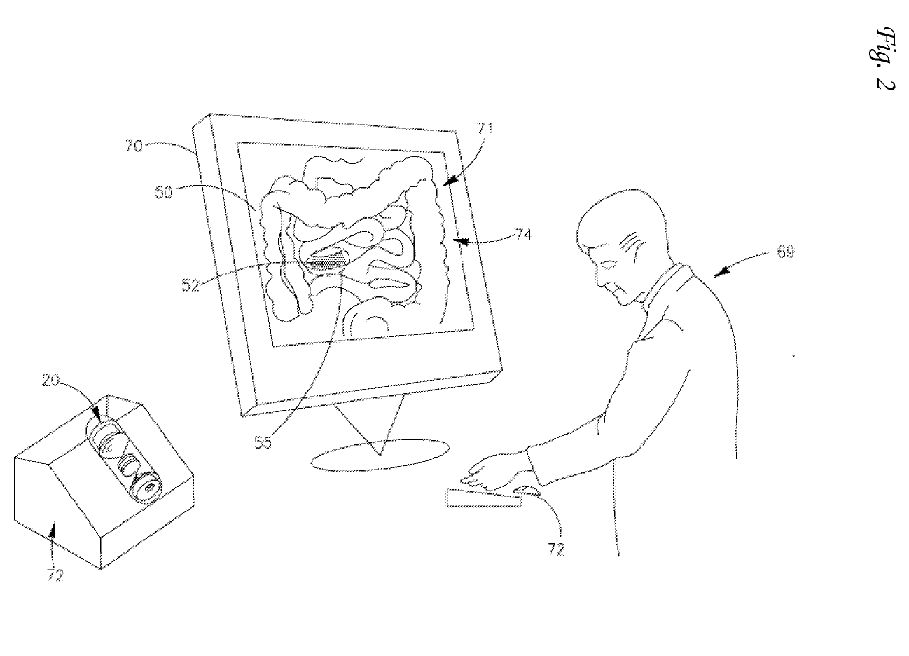

Photopill 20 may be programmed with phototherapy start and stop times to provide phototherapy to a diseased region of a patient's GI tract optionally using a personal computer (PC). By way of example, FIG. 2 schematically shows a medical professional 69 programming photopill 20 using a PC 70 having a monitor 71, in accordance with an embodiment of the invention. PC 70 is connected by a wire or wireless communication channel with a docking station 72 in which the photopill is seated for programming. Docking station 72 optionally communicates with photopill 20 using Bluetooth to transmit commands from PC 70 to the photopill.

In an embodiment of the invention, the medical professional displays an image 74 of the patient's GI tract 50 on the PC's monitor 71 with the diseased region or regions highlighted or otherwise indicated. By way of example, in image 74, a diseased region 52 of the patient's GI tract, 50 is schematically highlighted by shading. The medical professional selects a region of GI tract 50 to be illuminated with phototherapeutic light from photopill 20 by selecting an image of the region in image 74. Selection of a region in image 74 may be done using any of various methods known in the art such as by using a mouse to highlight the region or surround it with a border, or if monitor 71 is a touch screen, by touching the region to be selected. In FIG. 2, by way of example, the medical professional uses a mouse 73 to draw an ellipse 55 to define a region that includes diseased region 52 for receiving phototherapy.

PC 70 optionally computes phototherapy start and stop times responsive to the location of the indicated region and patient data relevant to speed with which photopill 20 is expected to travel through the patient's GI tract 50. The calculated phototherapy start and stop times are communicated from PC 70 to docking station 72, which transmits programming signals to photopill 20 to program the photopill with the start and stop times.

For photopill 20 comprising an accelerometer (or optical motion sensing means) that provides data for determining distance in the GI tract traveled by the photopill, PC 70 is used to program controller 26 to turn on and turn off light source 21 and/or light source 22 when distances traveled by the photopill determined from accelerometer output are equal to distances along the GI tract that bracket diseased region 52. Optionally, the distances that bracket the diseased region are locations at which ellipse 55 crosses a region of the GI tract enclosing the diseased region.

For a photopill comprising a pH monitor, in accordance with an embodiment of the invention, the photopill is optionally controlled responsive to pH in the GI tract. It is known that different portions of the GI tract are characterized by different pH values, and the photopill is programmed to turn on and provide phototherapy to a diseased region of the GI tract at a start time at which it reaches a region of the GI tract having a pH value characteristic of a portion of the GI tract in which a diseased region is located.

In addition to programming photopill 20 with phototherapy start and stop times, medical professional 69, optionally, programs photopill 20 with a desired intensity, and/or wavelength of phototherapeutic light to be applied to diseased region 52. For example, in embodiments of the invention for which light source 21 and 22 are tunable, or for which light source 21 provides therapeutic light in a wavelength band different form that of light source 22, the medical professional can also program photopill 20 to deliver different combinations of wavelengths of therapeutic light to diseased region 52. In some embodiments of the invention, the medical professional determines intensity of light provided by light sources 21 and 22 responsive to a total desired amount of therapeutic light to be deposited in diseased region 52.

As discussed hereinabove, the photopill capsule of the present invention may be programmed in order to control the activation and deactivation of the therapeutic light source, thereby ensuring that therapeutic light irradiation occurs at the desired site within the GI tract, as well as preventing unnecessary irradiation of non-target sites and premature depletion of the capsule battery.

In order to implement the programmable functionality, the capsule may, in one embodiment, comprise a programmable microprocessor controller which is small in size, low in current consumption, does not require external components to be operated and can function within the wide range of operating voltage supplied by the capsule's battery.

One preferred example of a microprocessor suitable for this task is MicroChip's PIC12F1822 processor, which is a self contained re-programmable controller, with a very small size (3×3 mm), and requiring no external components for its operation. This microprocessor can operate at voltages range from 1.8V to 5V. The PIC12F1822 has very low power consumption and contains digital inputs/outputs as well as several analog inputs for sampling and signal processing.

As discussed hereinabove, the photopill capsule may be used in one or more of several different activation/deactivation modes:
a) Timer based activation/deactivation
b) Location based activation/deactivation
c) pH based activation/deactivation

Timer Based Activation

This mode of operation will mostly be used for conditions where relatively predictable intestine travel speeds prevail, such as in clinical studies where patients are selected carefully according to pre-defined profiles, and are using the capsule in well controlled and monitored environment.

The capsule is triggered once it is removed from its package or by the patient before swallowing. Once triggered, the internal processor counts the time elapsed from trigger and once the pre-defined time-delay value is reached the capsule is activated (i.e. the therapeutic light source is turned on).

The pre-defined delay value reflects stomach delay time (for example—half an hour in a controlled environment) and additional delays to allow the capsule to reach its treatment target area (for example—for a 3 hour intestinal travel time, a 2 hour delay is required to reach the terminal ileum area).

Location Based Activation

This mode is suitable for use in cases where no prediction of intestine travel time exists, or where large variations are expected in travel speed values.

In order to use location based activation, it is generally necessary to use one or more of the mechanisms for determining the position and progress of the capsule within the GI tract, such as the accelerometer and optical position sensing means, described hereinabove. Using these mechanisms, it is possible to measure the progress of the capsule within the small intestine and to provide a momentary average travel speed, which if integrated, produces the distance traveled.

By knowing the distance traveled, the capsule can now be set to be activated at an absolute location within the small intestine (such as −4.5 meters beyond the pyloric sphincter) regardless of the time it takes to get there. It should be noted that this method is not particularly accurate, due to variable delays in stomach transit and also due to measurement errors.

pH Based Activation

Since pH values change significantly along the GI tract, the location of the capsule can be obtained by means of measuring the pH in the region in which the capsule is currently located. The following table provides the minimum and maximum pH values usually found in the various regions of the GI tract in healthy human subjects:

| | Min | Max |
|---|---|---|
| Location | pH | pH |
| Stomach | 1.0 | 2.5 |
| Proximal small | 6.1 | 7.1 |
| Intestine | | |
| Terminal ileum | 7.1 | 7.9 |
| Caecum | 6.0 | 6.8 |
| Left Colon | 6.3 | 7.7 |

It may thus be appreciated that the ambient pH value can be used to identify the entry of the capsule into the small intestine. From that point onwards, other mechanisms (such as the accelerometer and optical motion sensor described hereinabove) can be employed in order to measure the change in location within the small intestine.

Measurement of pH can be performed using a pH sensor incorporated within the capsule. While any suitable sensor can be used, in one embodiment, the pH sensor may be an ISFET (ion sensitive field effect transistor) sensor, such as the sensor used in the telemetry capsule described in US 2004/0106849.

In some embodiments of the present invention, the onboard microprocessor is also used for purposes other than activation/deactivation of the therapeutic light source, including (but not limited to) control of light source output intensity and calculation of speed and location parameters.

The actual setting of the programmable parameters in the capsule can be achieved by:
i) Pre-defined settings during manufacture of the capsule.
ii) Remote programming by the physician prior to use.

Manufacturing setup is a method whereby operating values are programmed into the capsule during the capsule manufacturing process. In particular, several versions of the microprocessor's software are prepared in advanced, each containing different setup (for example—one version might include delayed operation of 0.5 hour while another might include 2 hours).

The microprocessors are programmed with the different software versions and are assembled into capsules which are now labeled in order to distinguish between the different versions.

The second approach involves re-programming the capsule prior to ingestion. Thus, in cases where special setup parameters are needed (for example—if the patient's small intestinal travel speed is unusually high or low), the physician can change the setup parameters of the capsule in order to take the unusual physiological parameters into account. The re-programming can be achieved by means of 2-way wireless communication with the capsule, where parameters can be read from the capsule and written back into the capsule.

Such wireless communication can be achieved using standard wireless protocols such as WiFi or Bluetooth, or alternatively, by means of optical transmission between the physician's computer and the capsule.

It is noted that whereas in the above description a photopill in accordance with certain embodiments of the invention is either pre-programmed with a stop time or, alternatively, may be de-activated in response to changes in pH or detected position within the GI tract, in other embodiments of the invention a photopill is not programmed with a stop time. Instead, a photopill once its light source is turned on continues to generate phototherapeutic light until its power source no longer has sufficient energy to power the light source.

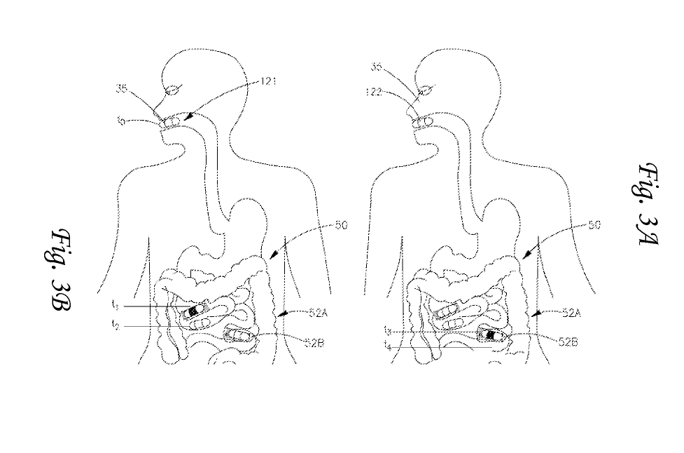

FIGS. 3A and 3B schematically illustrate photopills 121 and 122 being used to apply phototherapy, in accordance with an embodiment of the invention, to the GI tract 50 of a patient afflicted with inflammatory bowel disease (IBD) in regions 52A and 52B of the tract indicated with shading. Photopills 121 and 122 are similar to photopill 20 shown in FIG. 1 and comprise light sources 21 and 22 configured to emit light optionally in wavelength bands centered at 660 nm and 850 nm having bandwidths of about 30 nm. In FIG. 3A photopill 20 is swallowed at or about a clock-on time t_{O} of its timer 27 (FIG. 1) and begins traversing the patient's GI tract 50. The photopill is programmed, optionally as shown in FIG. 2, to turn on both light sources 21 and 22 at a phototherapy start time t₁ following t_{O} at which it is estimated it will reach diseased region 52A and to maintain the light sources on until a phototherapy stop time t₂ at which it leaves the region.

Photopill 121 is shown at various locations along GI tract 50 as it traverses the tract, and estimated locations of photopill 121 at times t_{O}, t₁, and t₂ are labeled with the times. The photopill's window 35 (FIG. 1), through which therapeutic light from light sources 21 and 22 is transmitted to illuminate annular regions of the tract, is shown unshaded to indicate when light sources 21 and 22 are off and is shown shaded to indicate when the light sources are on. In diseased region 52A, light sources 21 and 22 are on, and window 35 is shown shaded. In accordance with an embodiment of the invention, photopill 121 is programmed to deliver a total amount of therapeutic optical energy to diseased region 52A in each of the wavelength bands centered at 660 nm and 850 nm equal to about 0.1-1 Joules/cm². To provide the desired energy deposition, photopill 121 illuminates diseased region 52A with intensity of light in each band equal to the desired energy deposition divided by a time that the photopill is in the vicinity of, and illuminating the diseased region.

By way of example, assume power supply 24 of photopill 121 (shown in FIG. 3A) does not have enough energy to provide therapeutic light to both diseased regions 52A and 52B, and the photopill is used to provide phototherapy only to diseased region 52A. Photopill 122, schematically shown in FIG. 3B is used to deliver phototherapy to diseased region 52B. Photopill 122 is swallowed at or about a clock-on time t_{O}* and is programmed to turn on its light sources 21 and 22 at a time t₃ following t_{O}*, at which time t₃ photopill 122 is expected to arrive in the vicinity of diseased region 52B. Photopill is programmed to maintain its light sources on after turning them on at time t₃ until a time t₄ when the photopill is expected to leave the vicinity of diseased region 52B. In FIG. 3B figure, photopill 20 is shown passing through diseased region 52A with its light sources off (window 35 clear) and with its light sources 21 and 22 (window 35 shaded) on in the vicinity of diseased region 52B.

In an embodiment of the invention, photopills 121 and 122 are packaged in a protective package 130 schematically shown in FIG. 4 after they have been programmed with their respective phototherapy start and stop times and removal of a photopill from the package sets the clock-on time of the photopill.

Optionally, package 130 is formed having sockets 132 into which photopills 121 or 122 are inserted and stably held. In accordance with an embodiment of the invention, controller 26 comprised in photopills 121 and 122 has a magnetically activated “clock-on switch” (not shown) that operates to set the clock-on time in the photopills and package 130 comprises magnets 134 that generate a magnetic field in the vicinity of each socket 132. After a photopill 121 or 122 is programmed, when it is first placed in a photopill socket 132 of package 130, the magnetic field generated by magnets 134 in the vicinity of the socket arms the magnetic clock-on switch in the photopill's controller 26. When the photopill is removed from its socket and distanced from the magnetic field in the socket, the magnetic field in the vicinity of the photopill decreases substantially. The decrease in the magnetic field activates the magnetic clock-on switch to set a clock-on time for the photopill.

In some embodiments of the invention, a photopill comprises a clock-on switch, which is mechanically operated to set a clock-on time for the photopill when it is removed from a package.

FIG. 5 schematically shows a photopill 150 seated in a socket 160 of a package 162, which mechanically operates a clock-on switch in the photopill to set a clock-on time for the photopill when it is removed from the socket. Photopill 150 optionally has an elastic wall region 152, shown shaded and hereinafter referred to as a “push-button 152”, which is depressed and subsequently released to operate the switch. Socket 160 is formed having a protuberance, referred to as a spur 161, which is configured to depress push-button 152 when photopill 150 is seated in the socket. After photopill 150 is programmed with phototherapy start and stop times, the photopill is seated in socket 160 so that spur 161 depresses push-button 152. Depressing the push-button arms the clock-on switch. When photopill 150 is removed from socket 160 push-button 152 is released and the clock-on switch is switched to set the clock-on time for photopill 150.

It is noted that photopills are not limited to having their clock-on times set by a magnetic field or mechanically. In some embodiments of the invention a photopill clock-on time is set by exposure of the photopill to light. Optionally, the photopill comprises a photodiode that generates a signal responsive to incident light. The photopill is packaged in a light tight sleeve or package. When removed and exposed to light, the photodiode generates a signal that causes the clock-on time to be set.

In some embodiments of the invention, a photopill is activated to provide phototherapy to a region of a patient's GI tract responsive to a signal, hereinafter a beacon signal, transmitted by a beacon transmitter mounted on the patient's body. FIG. 6 schematically shows a photopill 170 comprising a controller 172 having a receiver, represented by an antenna 174, for receiving a beacon signal. Optionally, photopill 170 comprises a configuration of light sources for emitting phototherapeutic light different from that comprised in photopill 170 shown in FIG. 1. Photopill 170 optionally comprises a plurality of light sources 176 symmetrically positioned along a circumference of a circle to directly illuminate an annular region of a region of a GI tract in which it is located, optionally through a window 35 of the photopill.

Optionally, controller 172 is configured to process a proximity beacon signal established or transmitted by a proximity beacon located on the body of a patient whose GI tract is to be treated by photopill 170 with phototherapy. The controller turns on light sources 176 as photopill traverses the patient's GI tract responsive to a signal received by receiver 174 from the proximity beacon that indicates that the photopill is in a near neighborhood of the transmitter and therefore located in a region of the GI tract intended to receive phototherapy.

Any of various types of signals may be suitable as a proximity beacon signal. For example, a proximity beacon, in accordance with an embodiment of the invention, may provide an ultrasound or radio frequency (RF) proximity beacon signal. In some embodiments of the invention, a proximity beacon generates a relatively constant field such as a magnetic field inside the patient's body. Controller 172 senses the field and determines when to turn on light sources 176 responsive to the strength of the sensed field.

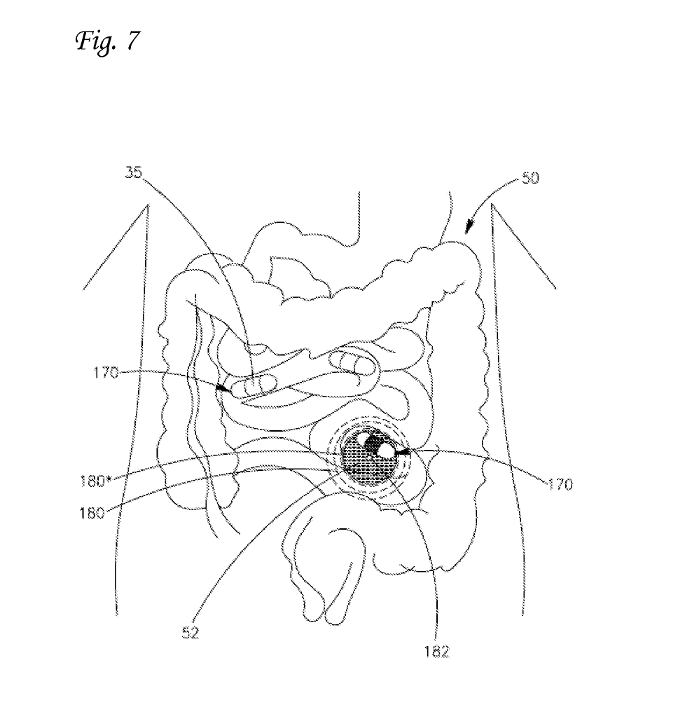

FIG. 7 schematically shows photopill 170 being used to provide phototherapy to a diseased region 52 of a patient's GI tract 50 responsive to beacon signals represented by dashed concentric circles 180, hereinafter referred to also as “signal circles”, transmitted by a beacon 182 located on the patient's body close to the diseased region. Intensity of beacon signals 180 decreases with distance from beacon 182. Photopill 170 is optionally programmed to turn on light sources 176 and maintain the light sources on as long as intensity of beacon signals 180 that receiver 174 receives is greater than a predetermined threshold intensity. A region in the patient's body at which beacon signal intensity is about equal to or greater than the predetermined signal strength is schematically indicated by an area within a solid “threshold circle” 180* concentric with “signal circles” 180.

In FIG. 7 photopill 170 is shown at various locations in GI tract 50 after it is swallowed by the patient. The photopill remains off (that is light sources 176 are off), as indicted by clear window 35 as long as it remains outside of threshold circle 180*. Once it reaches threshold circle 180* and remains within a region of the patient's body under the circle's area, beacon signals 180 that photopill 170 receives have intensity greater than the predetermined threshold intensity and the photopill is “on” and delivers therapeutic light to diseased region 52 of GI tract 50. The on state of photopill 170 within circle 180* is indicated by shading of its window 35.

In some embodiments of the invention, a photopill similar to photopill 170 processes directional beacon signals transmitted by a directional beacon to determine locations of the photopill in a patients GI tract and determine when to turn on its light sources and provide phototherapy to the GI tract.

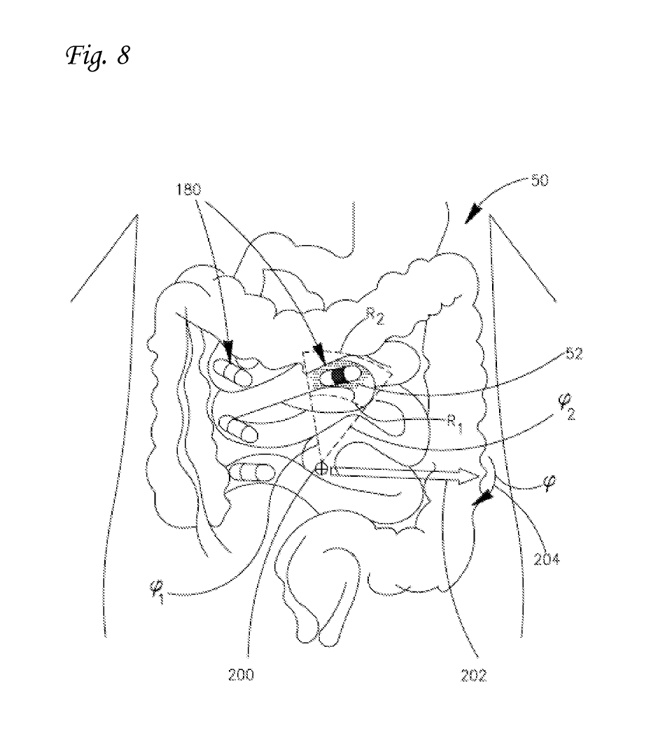

FIG. 8 schematically shows a photopill 180 providing phototherapy to a diseased region 52 of a patient's GI tract 50 responsive to directional beacon signals that the photopill receives from a directional beacon 200 mounted at a known location on the patient's body. By way of example, directional beacon 200 is shown mounted to the patient's body in a region of the navel.

Directional beacon 200 transmits a rotating beam, represented by a block arrow 202 of optionally acoustic energy, whose frequency “f” and intensity “I” change respectively with an angular direction “φ” along which the beam is transmitted and a radial distance “r” in the patient's body from the beacon. Angular direction φ of beam 202 is an azimuth angle about an axis (not shown) that passes through beacon 200 and is perpendicular to the coronal plane of the patient's body (an axis perpendicular to the page of FIG. 8). Direction of rotation is optionally clockwise and indicated by a curved arrow 204. Frequency f and intensity I are written f(φ) and I(r) to explicitly show their respective dependence on azimuth angle and radial distance relative to directional beacon 200.

A location of a region in the patient's body may be determined relative to the position of directional beacon 200 by determining a frequency and intensity of beam 202 at the location. For example, a lookup table may be used to map a frequency f(φ) and intensity I(r) of beam 202 measured at a given location in a patient's body to the azimuth angle φ and radial distance r coordinates of the location. In FIG. 8, diseased region 52 is schematically shown located between azimuth angles φ₁ and φ₂ and radial distances r₁ and r₂ relative to directional beacon 200. In the figure, dashed lines labeled respectively φ₁ and φ₂ bracket the angular extent of diseased region 52 and dashed lines labeled r₁ and r₂ respectively bracket the radial extent of the diseased region. In an embodiment of the invention, the diseased region is associated with corresponding frequencies in a range of frequencies between frequency f(φ₁) and frequency f(φ₂) and corresponding intensities between I(r₁) and I(r₂).

After photopill 170 is swallowed and travels along GI tract 50 its receiver 174 (FIG. 6) receives directional beacon signals 202 transmitted by directional beacon 200, which signals are processed by controller 172 to determine their frequency and intensity. Upon receiving directional signals having frequency and intensity in the ranges f(φ₁)-f(φ₂) and I(r₁)-I(r₂) that mark the location of diseased region 52, the controller turns on light sources 176 to illuminate the diseased region with phototherapeutic light.

It is noted that whereas in the above description a photopill provides light to a diseased region of the GI tract, a photopill in accordance with an embodiment of the invention is not limited to providing phototherapy to a diseased region of the GI tract. A photopill may for example be used to illuminate a portion, or substantially all of a patient's GI tract, to provide preventive therapy to the patient.

The photopill capsules of the present invention may be used by a patient to treat conditions in the gastrointestinal tract in the following manner:

The photopill capsule may be self-administered by the patient after waking up in the morning, at least half an hour before eating. Alternatively, it may be taken at least 4 hours following food consumption (fluids can be consumed at any time). This requirement ensures that the stomach is empty and remains empty until the capsule travels away from the stomach into the small intestine.

The photopill capsule is generally packed in a 6 or 10 capsule package. Before usage, the user removes the capsule from its package.

Once removed from its package, the capsule is activated (by means of one of the activation modes described hereinabove, and indicated by a visible red light flashing once a second, 3 times, from within the capsule) and should be swallowed within 5 minutes of removal from package. The user should verify that the capsule is active. In another version of the capsule, activation may be achieved by squeezing the capsule (as described above). In this context, activation of the capsule refers to the activation of a timer, thereby placing the capsule in a state in which it is ready to be swallowed by the patient. It should be noted that the therapeutic light source will be turned on later on, in response to signals generated by the timer, accelerometer or optical detector.

The following examples are provided for illustrative purposes and in order to more particularly explain and describe the present invention. The present invention, however, is not limited to the particular embodiments disclosed in these examples.

### Example 1

### Typical Phototherapy Capsule of the Present Invention

Physical dimensions—Length 11 mm

External diameter 27 mm

The outer shell is transparent and is made of a mixture of Polycarbonate, Polystyrene and K-resin with a wall thickness of 0.4 mm, and is manufactured using a conventional molding technique as well known to the skilled artisan . . . .

The power source contained within the capsule is a small cylindrical battery—GP1015L08—having a length of 15 mm and a height of 10 mm.

The capsule includes 2 electronic printed circuit boards, the first of which is a DC to DC driver (Texas Instrument's TPS61041) used to drive the LEDs used in the capsule, and a controlling microprocessor (MicroChip's PIC12F1822-I/MF) which controls the activation and operation of the capsule.

The second circuit hosts the Photo-therapy LEDs in a circular arrangement, such that the light generated by said LEDs is transmitted radially outwards.

The LEDs are UT-692UR supplied by L.C LED and provide light centered at 660 nm.

As part of the capsule's shell, above the LEDs, is a beam-shaping optics which re-shapes the LEDs radiated energy into a uniform “ring” shaped beam around the capsule. The optics will be incorporated into the capsule's transparent shell and will be designed as a “ring” surrounding the area of the LEDs. The optics is designed to concentrate the beam on the capsule's longitude axis while not affecting the radial axis beam. The capsule also comprises a small accelerometer (ADXL337 manufactured by Analog Devices) connected to the above-mentioned controlling microprocessor.

### Example 2

### Effect of Intraluminal Phototherapy in a Murine Colitis Model

Introduction:

A dextran sulfate sodium (DSS)-induced colitis model in mice was used to demonstrate the positive therapeutic effect obtained by using intraluminal phototherapy to treat inflammatory lesions of the GI tract. Colitis was induced in C57BL/6 mice by adding DSS to their drinking water, in accordance with standard protocols for chronic and acute DSS-induced colitis [Wirtz et al., 2007, Nature Protocols Vol. 2 pp. 541-546]. Phototherapy treatment was carried out using a Storz mini-endoscope system fitted with intraluminal light sources emitting at 440±40 nm (blue), 660±50 nm (red) and 850±50 nm (near infra-red [NIR]).

Severity of the induced colitis was assessed endoscopically using the following set of criteria:

Murine endoscopic index of colitis severity

| | 0 | 1 | 2 | 3 | Total |
|---|---|---|---|---|---|
| Thickening | Transparent | Moderate | Marked | Non- | 0-3 |
| of the colon | | | | trans- | |
| | | | | parent | |
| Changes of | Normal | Moderate | Marked | Bleeding | 0-3 |
| the vascular | | | | | |
| pattern | | | | | |
| Fibrin visible | None | Little | Marked | Extreme | 0-3 |
| Granularity | None | Moderate | Marked | Extreme | 0-3 |
| of the | | | | | |
| mucosal | | | | | |
| surface | | | | | |
| Stool | Normal + | Still | Unshaped | Spread | 0-3 |
| consistency | solid | shaped | | | |
| | | | | | Overall: |
| | | | | | 0-15 |

Experimental Design:

acute 2% DSS colitis was induced in 44 mice, which were then allocated to one of four treatment groups (each containing 11 mice): two treatment groups (A and B), sham treatment (no light source used) and control, in accordance with the protocol shown in the following table:

| | Duration of each | Frequency of | Light source | Irradiation |
|---|---|---|---|---|
| | phototherapy | treatments | wavelength | intensity |
| | session (min.) | (session/week) | (nm) | (j/cm2) |
| A | 7 | 2 | 820 | ~1 |
| | | | (NIR) | |
| B | 3.5 | 2 | 660 | ~1 |
| | | | (red) | |
| Sham | 3.5 | 2 | No light | — |
| Control | No | — | — | — |
| | phototherapy | | | |

During the treatment phase (groups A, B and sham), the colonoscope was inserted into the colon as far as the splenic flexure, and the pulled out gradually to simulate the movement of an ingested capsule device. The following table presents the results for disease severity assessed at three different time points (measured from day zero of the study)—Day 9, Day 13 and Day 19:

| Group | Day 9 | Day 13 | Day 19 |
|---|---|---|---|
| 850 | 7.29 ± 0.76 | 9.57 ± 2.30 | 9.60 ± 2.07 |
| 660 | 7.60 ± 1.14 | 9.20 ± 2.59 | 9.60 ± 2.30 |
| Sham | 10.00 ± 1.26 | 11.50 ± 2.07 | 11.00 ± 0.63 |
| Control | 9.60 ± 0.89 | 12.40 ± 2.07 | 12.00 ± 1.00 |

When the results for treatment groups A and B were taken together, the reduction in disease severity in the phototherapy-treated animals in relation to the sham group was statistically significant at each of the three time-points.

The results of this study demonstrate that intraluminal phototherapy is effective in significantly reducing the severity of colitis in a mouse model.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A swallowable capsule for providing phototherapy to a region of a patient's gastrointestinal (GI) tract, the capsule comprising:
one or more light sources emitting in the visible and/or NIR ranges; and
optical elements for shaping the light beam produced by said light sources, such that said light source(s) and said optical elements are capable of delivering an effective therapeutic dose to a target site within the GI tract.

2. The swallowable capsule according to claim 1, wherein the light radiation produced by the light source(s) is transmitted out of said capsule in a direction that is essentially perpendicular to the longitudinal axis thereof, and wherein said transmitted light is in the form of an essentially circular narrow band of light surrounding said capsule.

3. The swallowable capsule according to claim 1, wherein the light sources emit light centered at one or more wavelengths in the ranges selected from the group consisting of 400-480 nm, 610-720 nm and 800-950 nm.

4. The swallowable capsule according to claim 3, wherein the light sources emit light centered at one or more wavelengths selected from the group consisting of 440 nm, 660 nm and 850 nm.

5. The swallowable capsule according to claim 1, further comprising means for determining the direction, speed of movement and location of said capsule.

6. The swallowable capsule according to claim 5, wherein the means for determining the direction, speed of movement and location of said capsule is selected from the group consisting of an accelerometer and an optical motion-sensing system.

7. The swallowable capsule according to claim 1, wherein said capsule further comprises one or more microprocessors and associated circuitry.

8. The swallowable capsule according to claim 1 wherein said capsule further comprises a timer.

9. The swallowable capsule according to claim 1, wherein said capsule further comprises a switch selected from the group consisting of a magnetic switch and a mechanical switch, and wherein said switch is capable of turning on one or more elements defined in the preceding claims selected from the group consisting of a timer, an accelerometer and an optical motion-sensing system.

10. The swallowable capsule according to claim 9, wherein the switch is capable of being activated upon removal of said capsule from a package.

11. The swallowable capsule according to claim 9, wherein the switch is a mechanical switch which is capable of being activated by means of squeezing the capsule.

12. The swallowable capsule according to claim 1, wherein said capsule further comprises means for detecting changes in the ambient environment of the capsule.

13. The swallowable capsule according to claim 12, wherein the means for detecting changes in the ambient environment of the capsule comprise a pH sensor.

14. A method for intraluminal phototherapy of the gastrointestinal tract in a patient in need of such treatment, wherein said method comprises the steps of:
a) providing a swallowable capsule according to claim 1; and
b) oral administration of said capsule to said patient.

15. The method according to claim 14, wherein the oral administration to the patient is by way of self-administration.

16. The method according to claim 14, wherein said method is used to treat inflammatory bowel disease.

17. The method according to claim 16, wherein the inflammatory bowel disease is selected from the group consisting of Crohn's disease, ulcerative colitis and indeterminate colitis.

18. The method according to claim 14, wherein said method is used to promote healing of the intestinal mucosa and submucosal tissues.

19. The method according to claim 14, wherein said method is used to treat lesions in the small intestine.

20. The method according to claim 14, wherein said method is used to treat mucosa in the small intestine.
